(19) United States
(12) Patent Application Publication
(10) Pub. No.: US 2010/0012118 A1
(43) Pub. Date: Jan. 21, 2010

(54) Medicament dosage for inhaler

(76) Inventor: Markus Storz, Tuttlingen (DE)

Correspondence Address: Charles H Jew, 236 West Portal Avenue, Suite 533, San Francisco, CA, 94127, US

(21) Appl. No.: 12/459,899

(22) Filed: Jul. 9, 2009

(30) Foreign Application Priority Data
Jul. 19, 2008 (EP) 08013064

(51) Int. Cl. A61M 15/00; A61K 31/352; A61K 9/70

(52) U.S. Cl. 128/203.15; 514/454; 424/443

(57) ABSTRACT

The manual filling of vaporizable substances into the filling chamber of an inhaler by the user is suitable, in particular, for the requirements of a medical application only in limited circumstances. As a rule, there is the risk of an incorrect dosage. Moreover, many patients are not at all capable of carrying out such a difficult task owing to their motor skills ailments or other conditions that render them disabled. Medical application therefore requires an economical form that has already been pre-dosed with the desired substance (medicament) by the pharmacist or the pharmaceutical company and that is a standardized single-use component. A dosage pad that has already been pre-dosed by the manufacturer ensures that the proper amount of medication is used. In particular, a dosage pad containing a medicament that is vaporizable by heat in an inhaler and that is effectual upon inhalation avoids the problems associated attempting to fill an inhaler manually with the correct dosage.

### FIELD OF THE INVENTION

The present invention generally relates to a dosage form containing a pre-measured amount of medicament that is used in an inhaler and in particular to a dosage pad that contains herbal cannabis, cannabis extract, cannabinoid or opioid.

### BACKGROUND OF THE INVENTION

A dosage form is the physical form of a dose of medication such as a capsule or injection. The route of administration is dependent on the dosage form for a given drug. An inhaler is a device for delivering medication into the body via the lungs. The most common is the pressurized metered-dose inhaler (MDI). In MDIs, medication is typically stored in solution in a pressurized canister that contains a propellant, although it may also be a suspension. The MDI canister is attached to a hand-operated actuator. On activation, the MDI releases a fixed dose of medication in aerosol form.

Conventional inhalers such as the MDIs are not suited for delivering medication that is present in plants and herbs. This is particularly the case where patients have been prescribed cannabis for treating various indications such as glaucoma, nausea, and other symptoms.

### SUMMARY OF THE INVENTION

The present invention is based in part on the development of a single-use dosage pad that incorporates therapeutic amounts of a medicament that contains vaporizable active substances. The dosage pad is particularly suited for use in an inhaler that has a filling chamber or receptacle for receiving the dosage pad and a source of thermal energy such as a hot air generator that heats the medicament which causes the release of the active substances as aromatic vapors. The vapors can be inhaled directly by the patient. The inhaler can also be equipped with a container such as a valve-operated balloon to capture the vapors so that the patient can inhale the medicament as needed. Because the manufacturer or the pharmacist does the pre-dosing, a benefit of the dosage pad is that patients do not need to be concerned with measuring the precise amounts of medicament for placement into the inhaler.

### BRIEF DESCRIPTION OF THE DRAWINGS

FIG. 1 shows an axial section through the dosage pad according to the invention with an herb or powder filling;

FIG. 2 shows an axial section through the dosage pad according to the invention with an absorbent material that is positioned between the layers and is for receiving liquid substances;

FIG. 3 shows an axial section through the dosage pad according to the invention with an absorbent material that is positioned between the layers and is for receiving liquid substances, as well as a protective packaging composed of a film that is impervious to light and that is necessary for packaging in a protective environment;

FIG. 4 shows an axial section through the dosage pad according to the invention with an absorbent material that is positioned between the layers and is for receiving liquid substances, positioned in a filling chamber that is provided for receiving pads; and

FIG. 5 shows a perspective view of an herb dosage pad.

### DETAIL DESCRIPTION OF PREFERED EMBODIMENTS

The present invention is directed to a medicament dosage form that is particularly suitable for use with an inhaler that generates aroma and/or active substance containing vapors from plant material such as herbal cannabis. Inhalers are described, for example, in U.S. Pat. No. 6,513,524 to Storz, which is incorporated herein. This inhaler serves to thermally vaporize for subsequent inhalation of vaporizable, that is to say, with the influence of heat, aerosol-forming substances. These substances are, according to the available prior art, either placed on their natural carrier, for example the plant fibers that cannot be vaporized of an herb or other plant parts, or in the case of liquid or pulverized substances, they are placed on an artificial carrier, for example, a pad composed of pressed stainless steel wire mesh in a filling chamber. This stainless steel wire mesh is provided for reusing, while the dosage must be applied by the user and the pad must be cleaned after use for the next application. The filling chamber, which is filled with plant matter or the stainless steel wire mesh and the substances placed thereon, has hot air flow through it thereafter by means of which the substances vaporize or evaporate and are converted into hot air. This air, which is mixed with aromas and/or active ingredients, is inhaled after it has cooled to a comfortable temperature, the active ingredients entering the bloodstream by means of the pulmonary alveoli.

The manual filling of the substances into the filling chamber by the user is not always suitable for the requirements of a medical application. As a rule, there is the risk of an incorrect dosage. Substances that are intended to be administered in the milligram range can rarely be weighed out reliably by a patient at home. If the substances are provided with bulking agents or solvents for better dosing and/or more even distribution on the stainless steel wire mesh, then it is necessary in the case of alcohol used as a solvent, for example, to vaporize it in advance at lower temperatures, that is to say, to bring about a separation, in order to subsequently make the desired substance vaporize so that inhalation can occur.

Due to motor skill ailments or other conditions that render patients disabled, many patients are not at all capable of carrying out such a difficult task. The present invention provides a medicament dosage form that contains a known amount of medicament. The invention prevents incorrect dosing and ensures a simple and reliable application of the inhaler.

This problem is solved according to the invention that, in one embodiment, includes a dosage of a medicament that is vaporizable by means of heat action and that is effectual upon inhalation, dosed for an application and manageably arranged as a component for use in an inhaler.

The dosage or its arrangement as a component or part can also be characterized as a pad or a dosage pad. This component could, for example, be a pocket, composed of two layers, in which the medicament is placed. Furthermore, it could be, for example, a substrate that is saturated with the medicament or contains it in some other way. The medicament could also be a powder that is contained in a fabric or sponge. Whatever the case may be, the component that contains the medicament can be handled manually, regardless of its design. For the sake of simplicity, this component is characterized as “dosage pad” in the description.

The subject matter of the invention is thus an economical component that has already been pre-dosed with the desired substance (medicament) by the pharmacist or the pharmaceutical company which, in other words, is to say that it is a one-piece object that can be picked up in the hands and that is intended for a single use in an inhaler.

The advantages derived from the invention consist particularly in that the filling of the filling chamber of an inhaler is substantially simplified for the user. Only one dosage pad needs to be inserted. Moreover, the risk of the user making an incorrect dosage is practically eliminated since professionals will have already supplied a correct dosage beforehand and the dosage pad can be designed for one-time applications. Likewise, it becomes unnecessary for a person to separate solvents of liquid substances.

An advantageous embodiment of the invention includes a dosage wherein the vaporizable substances are located in a layer of a material that is permeable to air and vapor. This pad could, for example, consist of a layer of a support material, which is in disk form, is absorbent, and is permeable to air and vapor, on which the substance has been supplied in a dosage amount by the manufacturers. The above-described separation would be unnecessary if liquid substances were dosed and sprayed on a pad without solvents by the manufacturers by means of a suitable apparatus. In using substances containing solvents on the pad, the manufacturers could already attend to the separation of the solvent prior to delivery to the user.

A further advantageous embodiment of the invention includes a dosage wherein the vaporizable substances are located between two protective layers of a material that is permeable by air and vapor and the circumferential edges of which are connected to each other. Solid substances such as, for example, pulverized plant matter or powder is placed between two layers of a material that is permeable by air and vapor and the circumferential edges of which are joined together.

Yet another advantageous embodiment of the invention includes a dosage wherein an additional layer, which is located between two protective layers of a material that is permeable by air and vapor, serves to receive liquid substances or solutions. Through the use of a substance support material between the two layers, it is possible, for example, for sticky/resinous substances to be vaporized in the dosage pad and to be stacked one atop the other, for example, without adhering to one another.

An additional advantageous embodiment of the invention includes a dosage that is packaged in a protective environment. By being packaged in a protective environment, air- and oxygen-sensitive substances can also be used for the dosage pad. Photosensitive substances can be protected with packaging that is impervious to light.

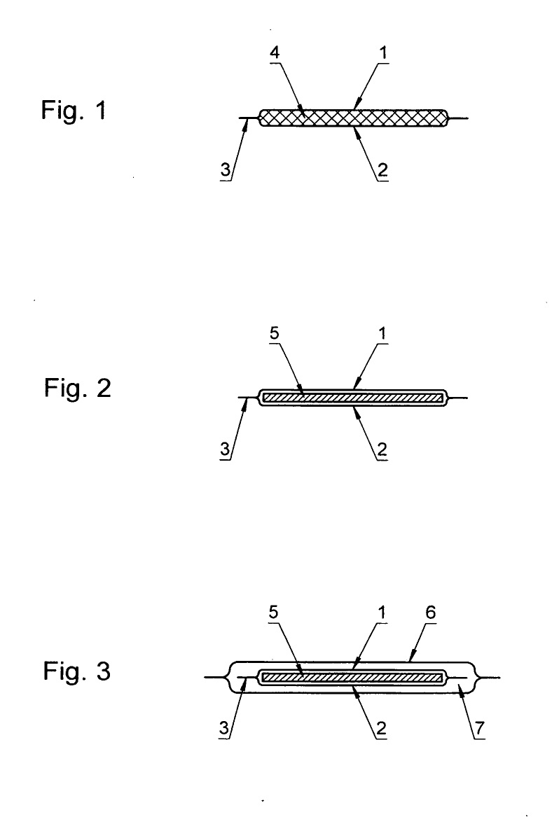

An illustrated representation of a one-layer, disk-shaped pad with applied substance dispensed with it can be readily presented. A dosage pad, when used for medicament that is herbal cannabis, cannabis extract, cannabinoid or opioid, is shown in FIG. 1 includes an upper layer (1) and a lower layer (2) of a material that is permeable by air and vapor such as tea bag paper, for example, and the edge regions (3) of which are connected to each other. The filling (4) of pulverized herbs or of other pulverized solid matter or powders is located there between.

The dosage pad which is characterized in that the vaporizable substances are located in a layer of a material permeable by air and vapor is shown in FIG. 2 has an upper layer (1) and a lower layer (2) of a material that is permeable by air and vapor such as tea bag paper, for example, and the edge regions (3) of which are connected to each other. An absorbent material (5) that is permeable by air, such as paper fleece, for example, is located there between for receiving liquid substances or solutions.

In FIG. 3, a dosage pad is shown as in FIG. 2, characterized in that an additional layer, which is located between two protective layers of material that is permeable to air and vapor, serves to receive liquid substances or solutions. In particular, the dosage pad is packaged under a protective gas (7) in an additional film (6) that is impervious to light such as an aluminum plastic composite layer film, for example.

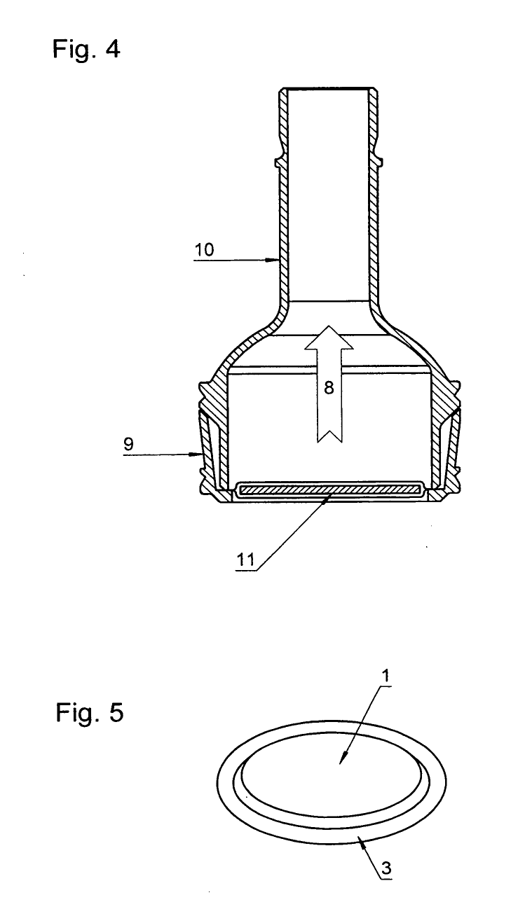

FIG. 4 shows a dosage pad according to the invention positioned in the filling chamber (9) provided therefor of an inhaler, which is customarily not shown, the screwed-on filling chamber cap (10) fixing the dosage pad on the location provided therefor at the correct distance from the heat source.

FIG. 5 shows a dosage pad according to the invention as shown in FIG. 2 in a perspective view.

The dosage pad of the present invention is particularly suited for administering active constituents, such as Δ⁹-tetrahydrocannabinol (THC) of the cannabis plant, which have been identified as effective in treating various ailments. For example, U.S. Pat. No. 4,189,491 to Shapiro et al. describes using THC in treating glaucoma. Similarly, U.S. Pat. No. 6,132,762 to Cristobal discloses the benefits of cannabis in treating joint pain, muscle pain, inflammation and arthritis. Finally, U.S. Pat. No. 6,974,568 to Piomelli teaches ameliorating or preventing cough by administering certain cannabinoid compounds. The disclosures of these patents are incorporated herein by reference.

The inventive dosage pad can incorporate therapeutic amounts of one or more active cannabis compounds that have been extracted from the cannabis plant or it can incorporate the cannabis plant itself. The amount of dried cannabis plant in each dosage pad typically ranges from 10 to 1000 mg and the amount of cannabis extract or pure cannabinoids in each dosage pad typically ranges from 1 to 100 mg. The amount of herbal cannabis will depend on the potency of the plant used. A physician can prescribe the dosage pad with the proper therapeutic amount of plant to treat a patient.

In use, the patient places the dosage pad in the filling chamber or receptacle of the inhaler as described in, for example, but not limited to, U.S. Pat. No. 6,513,524 to Storz. Activation of the inhaler heats the plant to vaporize the aromatic substances as well as the active ingredients into a container such as a valve balloon; the valve is connected during vaporization to a filling chamber, respectively to a mouthpiece during inhalation. The patient then inhales the vapor after being cooled as needed.

### REFERENCE NUMERAL LIST

1 upper layer
2 lower layer
3 edge region
4 herb or powder
5 absorbent substance
6 film casing
7 protective gas zone
8 direction of through flow
9 filling chamber
10 filling chamber cap
11 dosage pad

The foregoing has described the principles, preferred embodiment and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of present invention as defined by the following claims.

What is claimed is:

1. A dosage of a medicament that is vaporizable by means of heat action and that is effectual upon inhalation, dosage for an application and manageably arranged as a component for use in an inhaler.

2. The dosage of claim 1 characterized in that the medicament has herbal cannabis, cannabis extract, cannabinoid or an opioid.

3. The dosage of claim 2 wherein the medicament is herbal cannabis.

4. The dosage of claim 3 wherein the amount of herbal cannabis in the dosage ranges from 10 to 1000 mg.

5. The dosage of claim 2 wherein the medicament is cannabis extract or cannabinoid.

6. The dosage of claim 5 wherein the amount of cannabis extract or cannabinoid in the dosage ranges from 1 to 100 mg.

7. The dosage of claim 1 characterized in that the vaporizable substances are located in a layer of material that is permeable to air and vapor.

8. The dosage of claim 1 characterized in that the vaporizable substances are located between two protective layers of material that is permeable to air and vapor and the circumferential edges of which are connected to each other.

9. The dosage of claim 1 characterized in that an additional layer, which is located between two protective layers of material that is permeable to air and vapor, serves to receive liquid substances or solutions.

10. The dosage of claim 1 characterized in that the dosage is packaged in a protective environment.

11. The use of a dosage as specified in claim 1 in an inhaler.

12. A method of treating pain in a human comprising the steps of:
providing a dosage pad containing a therapeutically effective amount of herbal cannabis;
inserting the dosage pad into the filling chamber of an inhaler;
vaporizing aromatics and active ingredients containing cannabinoid from the herbal cannabis;
introducing the aromatics and active ingredients into a container; and
inhaling the aromatics and active ingredients from the container.

13. The method of claim 12 characterized in that the herbal cannabis is located in a layer of material that is permeable to air and vapor.

14. The method of claim 12 characterized in that the vaporizable substances are located between two protective layers of material that is permeable to air and vapor and the circumferential edges of which are connected to each other.

15. The method of claim 12 wherein the amount of herbal cannabis in the dosage pad ranges from 10 to 1000 mg.
